(19) United States
(12) Patent Application Publication
(10) Pub. No.: US 2019/0104744 A1
(43) Pub. Date: Apr. 11, 2019

(54) COMPOSITION COMPRISING AN ACTIVATED MICROBIAL BIOMASS

(71) Applicant: LASAFFRE COMPAGNIE, Paris (FR)

(72) Inventors: Pascal Lejeune, Tourcoing (FR); Jean-Bernard Souici, Estaires (FR)

(21) Appl. No.: 16/209,544

(22) Filed: Dec. 4, 2018

Related U.S. Application Data
(62) Division of application No. 14/365,700, filed on Jun. 16, 2014.

(30) Foreign Application Priority Data
Dec. 16, 2011 (FR) 1103902

(51) Int. Cl. A21D 8/04; A61K 35/745; A23L 33/14; A61K 35/741; A61K 35/744; A61K 36/064; C12N 1/04; A23L 33/135; A61K 35/747

(52) U.S. Cl. CPC A21D 8/045; A21D 8/047; A61K 35/745; A23L 33/14; A61K 35/741; A61K 35/744; A23V 2002/00; C12N 1/04; A23L 33/135; A61K 35/747; A23Y 2240/00; A23Y 2220/17; A61K 36/064

(57) ABSTRACT

The present invention relates to a composition comprising a substrate capable of being evenly coated with a microbial biomass, wherein said biomass represents from 10% to 30% by dry matter of the total dry matter of the coated substrate. It also relates to a method for preparing said composition and to a starter fermentation activator and to a probiotic comprising said composition.

The invention relates to the field of food compositions containing a microbial biomass, to a process for preparing same and also to fermentation activators or starters, and more particularly to the field of bread-making, the dairy industry and/or food supplements of “probiotic” type containing such compositions.

The present invention relates generally to an improved composition produced by means of a process for spraying a high level of microbial biomass, in particular of bacterial biomass, onto a substrate.

Said substrate of the composition according to the invention is in particular a substrate of granular type (granules or spherules, for example), specially capable of maintaining a high viability of the microbial biomass and good preservation of this viability over time (preservation/persistence of the viability). Said composition, which contains live microorganisms, in particular bacteria, sprayed onto said substrate, under the usual working conditions of the art, is generally used as a starter/leaven in dry powder form.

The technical field targeted in the present invention is in particular that of a composition dried according to an improved process suitable for the production of a live microbial biomass in dry form using a substrate of granular type capable of being evenly coated with a concentrated microbial biomass, it being possible for said biomass to represent from 10% to 30% by dry matter of the total dry matter of the coated substrate.

The two major techniques for preserving live bacterial biomass in dry form are lyophilization and spraying onto a substrate.

EP 0636692 A1 by the applicant describes a “Stable biomass based on yeast cells and lactic acid bacteria and process for its preparation”.

Lyophilization is a complex expensive technique and the drying yields are sometimes low depending on the strains and techniques used.

The spraying of bacteria onto a substrate is also known from EP 0 636 692 A1. However, these techniques remain limited with regard to the amount of biomass that can be deposited on the substrate [less than 0.5% DM W/W in the case of EP 0 636 692 A1; above this value, agglomeration (caking) of the powder and a loss of viability during storage are noted], which consequently limits the performance levels of the prior compositions thus produced.

The microbial biomasses sold today as starters for leavens are obtained either by spraying, or by mixing a large amount of lyophilisate and of yeast granules, or by mixing a large amount of lyophilisate with a diluent substrate, and they are stored in the absence of oxygen at −20° C., so as to have a lifetime of 2 years. In the absence of these storage conditions, the shelf life of the starters does not exceed 3 months.

The mixing operations, the need for an oxygen-free atmosphere, the use of a “quadriplex” leaktight packaging film and the method of storage are all parameters which still remain difficult to control, in certain countries in particular with regard to observation of the cold chain. This leads to problems of quality, without mentioning questions of industrial and environmental costs to be taken into account.

Another problem also arises, which is recurring and is linked to the current form of starters for leavens, namely that of the existence of a long lag time during the inoculation of the flour with the leaven, causing an extension of fermentation times, a variability of the duration of the leaven and a risk of development of the uncontrolled flora of the flour.

All of these problems limit the development of the application of starters/leavens in bread-making, although their use makes it possible to produce breads of high aromatic and nutritional quality.

More recently, it has also been possible to use live bacterial biomass as a probiotic, with the problems linked, on the one hand, to the need for a high concentration of live biomass and, on the other hand, to the survival of the microorganisms after passing through the stomach and its strongly acidic pH.

The inventors have, moreover, observed that:
the prior art compositions exhibit a heterogeneity owing to the presence of agglomerates which make these agglomerate-forming compositions difficult to handle;
the prior compositions have long lag times leading to long leaven times, typically from 16 h to 24 h, therefore requiring the use of large equipment for carrying out these fermentations; these long times substantially increase, moreover, the risk of development of an undesirable flora that may generate parasitic flavors, bread-making problems, or even the development of pathogenic bacteria;
the prior compositions as described in patent EP 0 636 692 A1 make it possible to reach, immediately after their production, and therefore without storage, a pH of 5.7 in 3 h in an acidification test, which is insufficient; an acidification so as to reach a pH typically below 5.4 after one year of storage at 20° C. is desired.

### SUMMARY OF THE INVENTION

The present invention intends to solve the problems mentioned above and to overcome the difficulties summarized here and encountered with the prior compositions.

The inventors have shown that there is in fact still the need for a composition of dry products based on a substrate which is capable of being coated and which allows a very high concentration of live bacteria to be sprayed thereon, it being possible for such a composition to be used in particular as a fermentation activator/starter or a probiotic.

Such a composition is improved in the sense that it must allow effective protection of the live bacteria sprayed onto said substrate with respect in particular to a series of “stresses” of the “temperature stress” type, the starter being stored at a positive temperature or even at ambient temperature—, of the “acid stress” type—in particular in the probiotic application, it corresponds to the resistance to the gastric pH—and of the “oxygen stress” type—during storage in air.

Thus, the present invention meets this longstanding need for a composition which has the qualities summarized above.

The subject of the present invention is therefore firstly a composition comprising a substrate capable of being evenly coated with a microbial biomass, which is preferably very highly concentrated, said biomass representing very high percentages of bacteria by dry matter of the total dry matter of the coated substrate.

The first subject of the invention is a composition comprising a substrate capable of being evenly coated with a microbial biomass, said biomass representing from 10% to 30% by dry matter of the total dry matter of the coated substrate.

Another subject of the invention is a process for preparing a composition, comprising the following steps consisting in:
i—introducing a substrate capable of being coated into a mixer through which an ascending stream of hot air passes,
ii—spraying a suspension of microbial biomass comprising more than 5% by dry matter of bacteria (WAN),
iii—drying by means of a stream of hot air, the temperature and the flow rate of which are fixed such that the temperature of said composition does not exceed 40° C.,
iv—recovering a coated substrate, and
v—obtaining said composition.

A subject of the invention is also a “Fermentation activator” or “Starter” containing the composition of the invention or as obtained according to the process of the invention, in particular of bread ferment type or of wine-making ferment type or else of milk ferment type.

Another subject of the invention is a “Probiotic” containing the composition of the invention or as obtained according to the process of the invention.

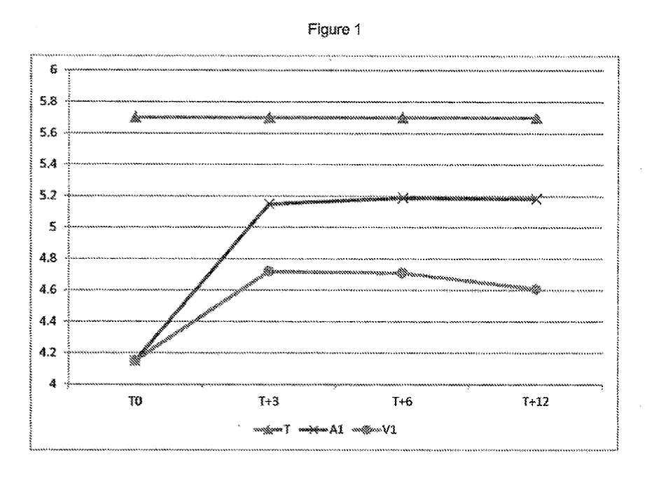

FIG. 1 represents the results of an acidification test carried out with a preferred composition of the invention, SPRAY_A, which was stored for 1 year at 20° C. under air (A1) and under vacuum (V1) in comparison with those obtained with a commercial controlled composition (T) stored under the best conditions, namely −20° C. under vacuum. FIG. 1 expresses the evaluation of the pH as a function of storage time in months.

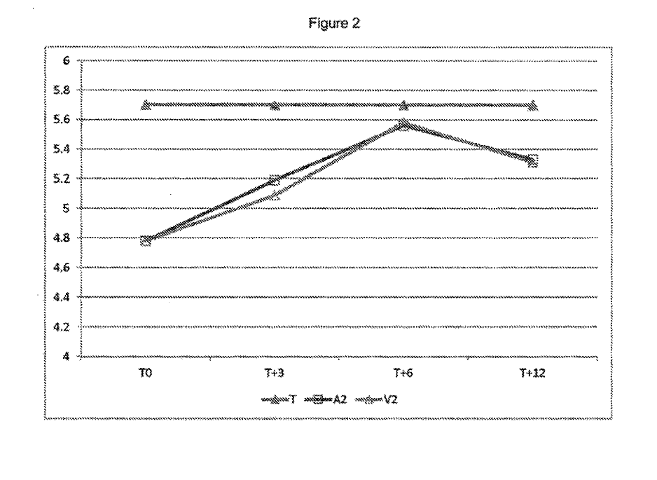

FIG. 2 represents the results of an acidification test carried out with a preferred composition of the invention, SPRAY_C, termed “overspraying” test, which was stored for 1 year at 20° C. under air (A2) and under vacuum (V2), in comparison with those obtained with a commercial control (T) stored under the best conditions, namely −20° C. under vacuum. FIG. 2 expresses the evaluation of the pH as a function of storage time in months.

### DETAILED DESCRIPTION OF THE INVENTION

The first subject of the invention is a composition comprising a substrate capable of being evenly coated with a microbial biomass, said biomass representing from 10% to 30% by dry matter of the total dry matter of the coated substrate.

The inventors of the present invention have particularly endeavored to develop an improved composition which contains a biomass which is stable and effective, and have developed a specific process for preparing said composition such that it favorably meets the particularly discriminating and significant criteria in the fields of application targeted and mentioned above.

The performance levels of the compositions according to the invention are controlled by means of specific tests, some of which have been developed by the inventors, and which have been used by the inventors during their research.

These tests, hereinafter referred to as “evaluation tests” are centered around the viability and the acidifying performance levels of the compositions, in particular:
the acidification test by observation of the decrease in the pH in 3 h of a medium based on maltose+mineral salts, carried out at 35° C. after inoculation of a predetermined amount of the composition;
the determination of the bacterial viability by measuring the number of bacterial colonies which develop on a standard medium by plating out a solution containing a known amount of the composition.

The time during which there is exposure to heat during the production of the composition of the invention is also a parameter which is taken into account.

The inventors have subsequently carried out measurements of the storage parameters for said composition after production thereof in accordance with the process of the present invention, thus defining percentages of preservation/persistence of the viability and acidifying performance levels.

A series of storage tests was thus carried out on the composition of the invention under various conditions, in particular temperature, air-exposure and duration. In particular, tests of the performance levels of said composition are carried out. These are in particular the following tests:
under air and under vacuum at −20° C. (reference storage temperature for this type of product),
under air and under vacuum at 4° C., and
under air and under vacuum at ambient temperature (20° C.).

The following were also determined:
the final amount of added dry matter of bacterial type in the composition per granule formed (DM bacteria/g of composition) (reminder: this amount depends on the spraying rate);
the final moisture content of the composition of the invention;
the particle size distribution (laser particle sizing) of the substrate of the composition according to the invention.

The composition of the invention has the following supplementary or alternative characteristics:
preferably, said biomass represents from 13% to 26% by dry matter of he total dry matter of the coated substrate;
the coated substrate advantageously also comprises a protective layer comprising at least one compound chosen from hydrocolloids, gums, dextrins, in particular maltodextrins, poly-, di- or monosaccharides, and derivatives thereof;
said substrate is preferably in the form of granules and/or of spherules;
said spherules preferably have a mean diameter d(0.5) of between 150 and 2000 μm, preferably between 150 μm and 1200 μm and even more preferentially between 150 μm and 700 μm;
said granules advantageously have a mean length of between 1.8 and 2.2 mm and a mean diameter of between 0.4 and 0.7 mm;
said substrate is advantageously chosen from active dry yeasts and cereal meals;
said yeasts preferably have a dry matter content of greater than 94%, preferably greater than 98%, said yeast granules advantageously having a beneficial effect as moisture absorber;
said meals preferably have a moisture content of less than or equal to 8%.

Advantageously, said yeasts are of the Saccharomyces genus comprising in particular the S. chevalieri species.

Preferably, said microbial biomass comprises at least bacteria chosen from the group consisting of bacteria belonging to one of the following genera: Lactobacillus, Pediococcus, Streptococcus, Leuconostoc, Lactococcus, Bifidobacterium, Propionibacterium and Bacillus.

Said bacteria are advantageously chosen from the group comprising: Lactobacillus plantarum, Lactobacillus brevis, Lactobacillus casei, Lactobacillus paracasei, Lactobacillus sanfrancisco, Lactobacillus amylovorurn, Lactobacillus kefir, Lactobacillus pentosaceus, Lactobacillus acidilactici, Lactobacillus rhamnosus, Leuconostoc oenos, Leuconostoc triesenteroides and Bacillus subtilis.

The coated substrate preferably also comprises a layer which consists of a cream yeast sprayed onto said substrate, preferably a cream of S. chevalieri, forming a protective layer. An improvement in the protection of the bacteria is then observed during the storage test under air.

The composition advantageously has a bacterial mortality rate of less than or equal to 0.5 Log CFU/g after storage for one year at a temperature of 20° C. under vacuum and/or after storage for one year at a temperature of 4° C. under air.

Preferably, the composition exhibits, after 1 year of storage at a positive temperature or even at ambient temperature, during the acidification test on medium comprising maltose, a decrease in pH from 6.5 to at least 5.7 after only 3 h.

Advantageously, the composition according to the invention, comprising a substrate which consists of active dry yeasts, also comprises at least on “drying” additive, or process aid, which is advantageously chosen from the group comprising modified fatty acid monoglycerides and diglycerides, fatty acid esters of sorbitan, such as sorbitan monostearate, fatty acid esters of glycerol, fatty acid esters of propylene glycol, methylcellulose, carboxymethylcellulose, hydroxypropylcellulose and/or a mixture of the latter.

A subject of the present invention is also a process for preparing a composition, comprising the following steps consisting in:
i—introducing a substrate capable of being coated into a mixer through which an ascending stream of hot air passes,
ii—spraying a suspension of microbial biomass comprising more than 5% by dry matter of bacteria (W/W),
iii—drying by means of a stream of hot air, the temperature and the flow rate of which are fixed such that the temperature of said composition does not exceed 40° C.,
iv—recovering a coated substrate, and
v—obtaining said composition,

The mixer which is used in step (i) is advantageously a fluidized air bed (FAB) which allows simultaneous spraying and drying, consequently providing protection by coating. Moreover, this process promotes better storage of the composition according to the invention, which is more stable over time, from the viewpoint in particular of better preservation of the viability of the biomass, event at ambient temperature.

The process according to the invention also has the o o supplementary or alternative characteristics:
preferably, the content by dry matter of bacteria sprayed in step (ii) of the process is between 10% and 26% and preferably between 13% and 26% by dry matter of the total dry matter of the composition (W/W);
steps ii and iii of the process are preferably simultaneous;
the process advantageously also comprises a step during which the coated substrate is covered by spraying with a cream yeast and/or with one or more compounds chosen from the group consisting of hydrocolloids such as gum arabic, locust bean gum, guar gum, gellan, xanthan, alginate or cellulose; starches such as native starch, pregelatinized starch or modified starches; dextrins such as maltodextrins; monosaccharides and disaccharides such as glucose, trehalose or sucrose; alone or as a mixture;
this “overspraying” by depositing a protective layer makes it possible to improve the storage under conditions under air and at 20° C.; in particular, the “oxygen stress” suffered by the composition of the invention is greatly reduced owing to this protection.

The following are also subjects of the present invention:
a “Fermentation activator” or “Starter” containing the composition of the invention or as obtained according to the process of the invention, in particular of bread ferment type, of wine-making ferment type or else of milk ferment type, and
a “Probiotic” containing the composition of the invention or as obtained according to the process of the invention,

In particular, the invention provides a composition with properties of interest that are particularly sought in the art.

Indeed, the research by the inventors has made it possible to develop a composition comprising a substrate which surprisingly makes it possible to increase the live microbial biomass which is sprayed onto said substrate so as to achieve a microbial, in particular bacterial, concentration never before achieved to date, said composition remaining stable over time. The inventors have also observed, surprisingly, a substantial improvement in the acidifying performance levels of said composition compared with the existing equivalent products, and also an excellent survival, even after 1 year of storage at ambient temperature or of storage under air.

### IMPLEMENTATION EXAMPLES

The present invention will now be described in detail in terms of its other characteristics and advantages by means of implementation examples given in a purely illustrative and nonlimiting manner and with reference to the appended tables and drawings.

### I—MATERIALS AND METHODS

Evaluation Tests

A]Acidification Tests with Maltose
1) Material and reagent:
   250 ml beaker,
   waterbath with stirring system, set temperature 35° C.,
   pH meter with recording device,
   medium maltose+salts:

| Ingredient | (g) |
|---|---|
| distilled water | 1000 |
| maltose•H₂O | 28.25 |
| K₂HPO₄ | 2 |
| MgSO₄•7H₂O | 0.37 |
| MnSO₄•H₂O | 0.055 |

If it is not used during the course of the day, this medium must be sterilized,
1N HCl.
2) Procedure, medium maltose+salts:
   Heat the waterbath to 35° C.
   Calibrate the pH-meter with the pH buffers and the temperature probes.
   Place 150±0.1 g of medium in the 250 ml beaker.
   Place the beaker in the waterbath, place a 25 mm magnetic bar in the beaker, and
   start the stirring at 500 rpm.
   Place the electrode of the pH-meter in the medium
   Weigh out the sample:
   weigh out 1 g of composition or 10 g of controlr 150 mi of medium.
   Start the timer and note the initial pH.
   After 5 min, add the sample as a fine rain while avoiding the formation of lumps.
   Wait ½ hour and add 1N HCl so as to decrease the pH to 6.20±0.05.
   Record the pH for approximately 20 hours and note the pH at t=3 h.

B] Viability Test
1) Material:
   Microbiological medium: M.R.S. agar from D FCO
   Sterilized actidione at 1.5%
   4% bromocresole purple solution
   Sterile 90 mm Petri dishes
   Incubator at 30° C.
   Gen-box anaerobic jars+sachets for anaerobiosis
   Sterile plastic pipettes
   Sterile spreaders.
2) Technique:
   Dilute 1 g of sample with qs sterile water to give 100 mL
   Homogenize well; this preparation is the 10⁻¹ dilution.
   Prepare successive dilutions down to 10⁻⁹ in tubes of sterile water.
   Plate out 0.1 ml at the surface on M.R.S. in Petri dishes with the appropriate dilutions.
3) Reading:
   Incubate the Petri dishes in an anaerobic jar, in an incubator at 30° C. for 24 to 72 h.
   Count the number of colonies which have appeared on the dishes and determine the number of colony-forming units (CPU) per ml (or per g) as a function of the dilution.

C] Measurement of the pH and of the T.T.A. (Total Titratable Acidity)
Place a sample of 10±0.1 g of crumb in a 250 ml beaker.
Prepare a volume of 100 ml of distilled water at ambient temperature.
Add approximately 40 ml of distilled water to the beaker and mix until homogenization (using a high-speed rotor/stator mixer of Ultraturrax type if necessary). Make up the volume with the rest of the water while using the latter to rinse the mixing instruments.
Add a magnetic bar and place the beaker on a stirrer plate.
Measure the pH (wait for stabilization of the pH which must last at least one minute).
Note the pH.
Using a 15 ml burette graduated to within 0.1 ml, run in an N/10 NaOH solution until the pH=6.6±0.1, wait 5 min, readjust the pH until stabilization at pH 6.6±0.1 for 1 minute.
Note the volume added in ml (=T.T.A.).

D] Commercial Control
Control: Saf Levain LV1, Lesaffre International S.A.R.L. 137 rue Gabriel Péri in 59700 Marcq-en-Baroeul, France.
This starter contains 5×10⁹ CFU of bacteria/g of Lactobacillus casei type.

E] Types of Granular Substrates
Instant dry yeast: Saf Instant, S. I. Lesaffre, 137 rue Gabriel Péri in 59700 Marcq-en-Barceul, France.
Fine clear durum semolina, biological (Moulin des moines Meckert-Diemer S.A. 101, route de Wingersheim in 67170 Krautwiller) superdried in a fluidized air bed dryer, by a batch of 25 kg for 20 min in an airstream of 1000 m³/h at 78° C.

F] Types of Bacteria
Lactobacillus casei: CNCM MA43/6V

### II—EXAMPLES

### Example 1

### Composition According to the Invention

### Example 1.1

### Preparation of a Cream of Bacteria

The Lactobacillus casei bacterial strain is propagated in a fermenter according to a conventional method which is moreover well known, using an M.R.S. medium for the precultures and the final culture, as described in “Bergey's Manual of systematic bacteriology—volume 2”, Sneath, P. H. A.; Main, N. S.; Sharpe, M. E. and Holt, J. G. (Eds), Williams and Wilkins (publisher), 1986, Baltimore.
At the end of fermentation, cell concentrations of approximately 10¹⁰ CFU/ml are obtained (CFU=number of cells able to reproduce per unit, in this case per ml); the fermentation must is then centrifuged so as to provide a cream of bacteria at approximately 20% total dry matter and approximately 1.5×10¹¹ CFU/ml.
This cream is stored in the cold (4° C.) while waiting to be used for the next step. This waiting time does not exceed a few hours.

### Example 1.2

### Preparation of the Coated Substrate by Spraying, According to the Process of the Invention, the Cream as Obtained in Example 1.1

The cream of bacteria of step 1 is sprayed onto the Instant Dry Yeast granular substrate and dried in a stream of hot air.
425 g of Saf-Instant yeast at 95.5% dry matter are introduced into a Glatt GPCG1.1 fluidized air bed. The apparatus is in the Wurster configuration and equipped with a 0.8 mm two-fluid spray nozzle in the bottom position.
690 g of cream of bacteria at 22.0% dry matter are thus deposited on the substrate and the apparatus operating parameters (flow rate and temperature of the fluidization air, flow rate of the spraying suspension) are chosen such that the temperature of the product, at any moment of the operation, is on average 39.2° C.
The duration of spraying and drying in the fluidized air bed is thus 124 min.

### Example 1.3

### Composition According to the Invention (Substrate+Bacteria)

Under the conditions described above, a final composition called “SPRAY_A” which has a moisture content of 5.3% and the bacterial content of which is 27.2% dry matter/total dry matter, is obtained.
Said composition initially contains, after drying, 5.0×10¹⁰ CFU of bacteria/g.

### Results

TABLE 1.a
Bacterial population of SPRAY_A during storage at 20° C.

| | Initial | 3 months | 6 months | 12 months |
|---|---|---|---|---|
| | CFU/g | CFU/g | CFU/g | CFU/g |
| Composition stored | 5.0E+10 | 1.9E+10 | 2.8E+10 | 1.1E+10 |
| under air | | | | |
| Composition stored | 5.0E+10 | 2.5E+10 | 8.0E+10 | 5.6E+10 |
| under vacuum | | | | |

The very small loss of live bacterial population during storage for 1 year at 20° C. during storage under air and the absence of loss of live bacterial biomass during storage under vacuum are noted in table 1.a—the apparent increase in the biomass during storage is an artifact linked to the uncertainty of the analytical method used.

As illustrated in FIG. 1, better acidification is observed with the “SPRAY_A” composition of the invention, even after storage for 1 year at a temperature of 20° C., in comparison with the commercial control stored under optimum conditions (−20° C. under vacuum), this being at equivalent bacterial biomass used.

As indicated in table 1.b, these results are particularly advantageous in comparison with those obtained with the control (Saf Levain LV1), under conditions of temperature stress (storage at 20° C.) or of oxidative stress (storage under air).

TABLE 1.b
Results of the SPRAY_A acidification
test during storage at 20° C.

| | Initial | 3 months | 6 months | 12 months |
|---|---|---|---|---|
| | pH | pH | pH | pH |
| | after 3 h | after 3 h | after 3 h | after 3 h |
| 10 g of control | 5.7 | 5.7 | 5.7 | 5.7 |
| 1 g of composition | 4.15 | 5.15 | 5.19 | 5.18 |
| stored under air | | | | |
| 1 g of composition | 4.15 | 4.72 | 4.71 | 4.61 |
| stored under vacuum | | | | |

They demonstrate the advantage of spraying a high level of bacteria according to the process of the invention compared with the known processes, since it improves the preservation of the five bacterial biomass and of its acidifying capacity under conditions of temperature stress or of presence of oxygen. It should be noted that similar results were obtained with a durum semolina.

### Example 2

### Preparation of an “Oversprayed” Composition According to the Present Invention Comprising a Coated substrate, Namely a Substrate and a Superficial Protective Layer

The cream of bacteria, obtained according to a procedure identical to that of step 1 of example 1, is sprayed onto the Instant Dry Yeast granular substrate and is dried in a stream of hot air. The resultant intermediate composition is then coated with a protective layer deposited by overspraying of a suspension (cream) of yeasts.

Step 1: Spraying of the Cream of Bacteria (SPRAY_B Composition)

The composition to be oversprayed is obtained under the following conditions:

935 g of cream of bacteria at 20.6% of dry matter are deposited on 600 g of Saf-Instant substrate. The operation is carried out in a fluidized air bed under the same conditions as in example 1.

A “SPRAY_B” composition to be oversprayed which has a moisture content of 5.2% and the bacterial content of which is 25.2% dry matter/total dry matter is obtained.

Step 2: Overspraying with a Cream Yeast (SPRAY_C Composition)

500 g of the preceding “SPRAY_B” composition are introduced into the Glatt GPCG1.1 fluidized air bed, which is in the Wurster configuration and is equipped with a 0.8 mm two-fluid spray nozzle in the bottom position.

235 g of cream yeast (Saccharomyces cerevisiae) at 24% dry matter are oversprayed onto the “SPRAY_B” composition and the apparatus operating parameters (flow rate and temperature of the fluidization air, flow rate of the spraying suspension) are chosen such that the temperature of the product, at any time during the operation, is on average 39.0° C. The duration of this operation is 37 minutes.

A composition “SPRAY_C” protected by a layer of yeast, the moisture content of which is 4.3% and which contains 22.5% bacterial dry matter/total dry matter, is finally obtained.

### Results

TABLE 2.a
Bacterial population of SPRAY_C during storage

| | Initial | 3 months | 6 months | 12 months |
|---|---|---|---|---|
| | CFU/g | CFU/g | CFU/g | CFU/g |
| Composition stored | 1.5E+10 | 1.5E+10 | 2.1E+10 | 1.6E+10 |
| under air at 4° C. | | | | |
| Composition stored | 1.5E+10 | 1.1E+10 | 1.4E+10 | 1.4E+10 |
| under vacuum at 20° C. | | | | |

The absence of significant loss of live bacterial population during storage for 1 year at 4° C. during storage under air and the absence of loss of live bacterial biomass during storage at 20° C. under vacuum are noted in table 2.a.

As illustrated in FIG. 2, better acidification is observed with the “SPRAY_C” composition according to the invention, even after storage for 1 year at a positive temperature (4° C.) in comparison with the commercial control, stored under optimum conditions (−20° C. under vacuum), this being at equivalent bacterial biomass used.

As indicated in table 2.b, these results are particularly advantageous in comparison with those obtained with the control (Saf Levain LV1), either under conditions of temperature stress (storage at 20° C.) or under conditions of oxidative stress (storage under air). These results demonstrate the advantage of overspraying as protection of the biomass of interest.

TABLE 2.b
Results of the SPRAY_C acidification test during storage at 4° C.

| | Initial | 3 months | 6 months | 12 months |
|---|---|---|---|---|
| | pH | pH | pH | pH |
| | after 3 h | after 3 h | after 3 h | after 3 h |
| 10 g of control | 5.7 | 5.7 | 5.7 | 5.7 |
| 1 g of composition | 4.78 | 5.19 | 5.56 | 5.33 |
| stored under air | | | | |
| 1 g of composition | 4.78 | 5.09 | 5.58 | 5.31 |
| stored under vacuum | | | | |

### Example 3

### Applications

### Bread Ferment

Production of a leavened bread from the composition in accordance with the invention and described in example 1.

Two bread-making tests are carried out according to the formula and the process described below.

Test 1: Leavened bread produced with the Saf Levain LV1 control at 3 months of storage under vacuum at −20° C.

Test 2: Leavened bread produced with the composition of example 1 at 1 year of storage under vacuum at 20° C. (ambient temperature).

1) Production of the Leavens:

Formula

| | Test 1: Leaven 1 | Test 2: Leaven 2 |
|---|---|---|
| Ingredients | % of flour used | % of flour used |
| T55 wheat flour | 100 | 100 |
| Water | 54 | 54 |
| Salt | 1.5 | 1.5 |
| Saf Levain LV1 | 0.5 | |
| Composition according | | 0.05 |
| to example 1 | | |

These two doughs, hereinafter referred to as “leaven” are left to ferment for 20 h at 30° C.

2) Production of the Breads:

Composition of the Final Dough

| | Test 1: Dough 1 | Test 2: Dough 2 |
|---|---|---|
| | % of flour used | % of flour used |
| T55 wheat flour | 100 | 100 |
| Water | 64 | 64 |
| Salt | 1.8 | 1.8 |
| Leaven 1 | 30 | |
| Leaven 2 | | 30 |
| Lesaffre Hirondelle bleue | 0.2 | 0.2 |
| pressed yeast | | |

Production Scheme
Artofex kneading

| | | |
|---|---|---|
| Slow speed (40 rpm) | 2 | min |
| Fast speed (60 rpm) | 13 | min |
| Dough temperature | 26° | C. |

Bulk fermentation

| | |
|---|---|
| Time | 160 min |
| Temperature | ambient |
| Humidity | ambient |

Weighing

| | | |
|---|---|---|
| Weight | 500 | g |
| Time | 10 | min |

Intermediate proofing

| | |
|---|---|
| Time | 20 min |
| Temperature | ambient |
| Humidity | ambient |

Proofing

| | |
|---|---|
| Time | 3 h |
| Temperature | ambient |
| Humidity | ambient |

Baking in a hearth oven

| | | |
|---|---|---|
| Time | 45 | min |
| Temperature | 225° | C. |

| | |
|---|---|
| Humidity | Steam: 1 + 1 |

3) Results:

| | Specific | | | |
|---|---|---|---|---|
| | volume | | Crumb T.T.A. | |
| | cm³/g | | ml (10 g) | Organoleptic |
| | cm³/g | Crumb pH | ml (10 g) | assessment |
| Test 1 | 4.1 | 4.4 | 3.4 | +++ |
| Test 2 | 3.9 | 4.3 | 4 | +++ |

These results, judged to be similar both in analytical terms and in tasting terms, show the advantage of the composition of example 1 compared with a commercial s control. Indeed, although stored for one year at ambient temperature and added at a dose 10 times lower than the control (test 1), the composition of example 1, in accordance with the invention, produces a bread with characteristics similar to those of a commercial starter stored for only 3 months under optimum conditions (−20° C. under vacuum).

1-17. (canceled)

18. A process for preparing a composition as claimed in one of claims 1 to 17, comprising the following steps consisting in:
i—introducing a substrate capable of being coated into a mixer through which an ascending stream of hot air passes,
ii—spraying a suspension of microbial biomass comprising more than 5% by dry matter of bacteria,
iii—drying by means of a stream of hot air, the temperature and the flow rate of which are fixed such that the temperature of said biomass does not exceed 40° C.,
iv—recovering a substrate capable of being coated, and
v—obtaining said composition,
wherein steps ii and iii are simultaneous.

19. The process as claimed in claim 18, characterized in that the content of bacteria is between 10% and 26% by dry matter and preferably between 13% and 26% by dry matter of the total dry matter of the composition (W/W).

20. (canceled)

21. The process as claimed in claims 18 or 19, characterized in that it also comprises a step during which the substrate is coated by spraying with a cream yeast and/or with a compound chosen from the group consisting of hydrocolloids such as gum arabic, locust bean gum, guar gum, gellan, xanthan, alginate or cellulose; starches such as native starch, pregelatinized starch or modified starches; dextrins such as maltodextrins; monosaccharides and disaccharides such as glucose, trehalose or sucrose; alone or as a mixture.

22. A fermentation activator of starter type obtained from a process as claimed in claims 18, 19 or 21.

23. A probiotic containing a composition obtained from a process as claimed in claims 18, 19 or 21.

24. The activator as claimed in claim 22, characterized in that it represents a bread ferment.

25. The activator as claimed in claim 22, characterized in that it represents a wine-making ferment.

26. The activator as claimed in claim 22, characterized in that it represents a milk ferment.
